(19) United States
(12) Patent Application Publication
(10) Pub. No.: US 2024/0338816 A1
(43) Pub. Date: Oct. 10, 2024

(54) Method for Recognizing Posture of Human Body Parts To Be Detected Based on Photogrammetry

(71) Applicant: MirEye Imaging Inc., Burnaby (CA)

(72) Inventors: Yifeng Zou, Sichuan (CN); Chuan Ma, Sechuan (CN)

(73) Assignee: MirEye Imaging Inc., Burnaby, BC (CA)

(21) Appl. No.: 18/575,592

(22) PCT Filed: Nov. 23, 2021
(86) PCT No.: PCT/CA2021/051667

§ 371 Date: Dec. 29, 2023

(30) Foreign Application Priority Data
Jul. 1, 2021 (CN) 202110740006.2

(51) Int. Cl. G06T 7/00; G06T 7/73

(52) U.S. Cl. CPC G06T 7/0012; G06T 7/73; G06T 2207/10028; G06T 2207/10116

(57) ABSTRACT

There is provided an alignment method of a human body part to be X-rayed based on photogrammetry. Preset positioning information of the target body part is input. An RGB camera and a depth camera capture natural images of the target body part. The spatial attitude deviation of the target body part is established relative to the spatial coordinate system where the X-ray machine tube is located. The present invention feeds back the difference between the patient's current posture and the theoretical best shooting posture to the medical staff in real time. The medical staff may instruct the patient to adjust the current position and posture, so that the patient's target position can be quickly adjusted to the theoretical optimal position range, providing a high-quality X-ray image in one shot, and the problem of low X-ray image quality caused by posture and distance problems can be eliminated.

### TECHNICAL FIELD

In general, this disclosure relates to the field of photogrammetry technology, in particular to the measurement of human body including spatial position, deflection angle and posture based on photogrammetry, and specifically to a method for recognizing the posture of a human body part to be detected based on photogrammetry.

### BACKGROUND

X-ray radiography is a very common inspection method in the medical field, particularly for bone parts, which can have a good image presentation. Radiography enables doctors to diagnose the target part of the patient using X-rays as a reference.

The quality of the X-ray image is the biggest factor that affects the doctor's judgment of the condition of the selected body parts. In addition to the equipment itself, the most important factor affecting the quality of the X-ray image is the center alignment, distance and angle between the selected body parts and the X-ray equipment (mainly the x-ray tube). In addition to the angle, when the existing X-ray image is taken, the patient's positioning can only be done based on an X-Ray operators' knowledge and experience, and there is no scientific basis and adjustment basis, nor can it be quantified. Therefore, if the existing X-ray image system wants to obtain the best quality at one time, the correct and precise body parts positioning is a must. If the quality of the X-ray image taken does not meet diagnostic requirements due to an incorrect or imprecise body parts positioning, it must be retaken, requiring the patient must be exposed to the radiation for the second time. During the retake, it is still possible for an incorrect or imprecise patient posture deflection leading to a second failure. The duplication of X-ray imaging causes patients and operators to receive excessive radiations.

The difficulties in existing X-ray image systems stem from the reliance of the system of the experience and visual inspection of medical staff, such that it is impossible to know the current posture and position of the patient's selected body part and the theoretical best shooting posture of the X-ray tube. The deviation between position of the patient's selected body part and the theoretical best shooting posture of the X-ray tube leads to the problem that the best X-ray image cannot be obtained.

### SUMMARY

One aspect of the present invention provides a method for real-time recognition of the current position and posture of the human body relative to the X-ray equipment (mainly referring to the x-ray tube), so that the medical staff can set the position correctly and precisely immediately before taking the X-ray. The deviation between the target part of the human body and the best imaging center position is used to guide the patient to adjust in real time, to take the X-ray as close as possible to the best posture and obtain the best or close to the best quality X-ray image at one time. The present invention reduces the current technology process of manually setting the center alignment between the selected body part and the x-ray tube based on the X-Ray operators' knowledge and experience and hence, increases workflow efficiency. In this manner, the patients and operators exposure to the soft radiations and excessive radiations in the X-Ray room is reduced, and the operator's direct contact with the patient is reduced.

In one aspect there is provided a method for posture recognition of a human body part to be imaged with an X-ray machine, comprising the steps of: inputting preset position information of the body part to be detected; adjusting the body part into a field of view of a camera; using the camera to capture a natural image of the body part; using a depth camera to capture a depth image of the body part; establishing a spatial attitude deviation of the body part relative to a spatial coordinate system where the X-ray tube is located; determining a desired position for the body part based on the spatial attitude deviation; providing the desired position for the body part in real time to an operator; and adjusting the body part to a new position based on the desired position.

In a further aspect there is provided an X-ray system for imaging a body part, comprising: an RGB camera aligned towards the body part and configured to capture a natural image of the body part; a depth camera aligned towards the body part and configured to capture a depth image of the body part; an X-ray tube aligned towards the body part;
a controller connected to the RGB camera and depth camera; wherein controller establishes a spatial attitude deviation of the body part relative to a spatial coordinate system where the X-ray tube is located, determines a desired position for the body part based on the spatial attitude deviation and provides the desired position for the body part in real time to an operator.

### BRIEF DESCRIPTION OF THE DRAWINGS

The invention will be further understood from the following description with reference to the attached drawings.

FIG. 1 is a schematic diagram of chest recognition of an example embodiment of the present invention.

FIG. 2 is a schematic diagram of chest sampling of an example embodiment of the present invention.

FIG. 3 is a schematic diagram of the normal direction of the space coordinate of an example embodiment of the present invention.

FIG. 4 illustrates a sample schematic of use of the invention in a scan of a chest of a human patient.

### DETAILED DESCRIPTION

The exemplary embodiments of the present disclosure are described and illustrated below for example purposes only. Of course, it will be apparent to those of ordinary skill in the art that the embodiments discussed below are exemplary in nature and may be reconfigured without departing from the scope and spirit of the present disclosure. However, for clarity and precision, the exemplary embodiments as discussed below may include optional steps, methods, and features that one of ordinary skill should recognize as not being a requisite to fall within the scope of the present disclosure.

One example aspect of the present invention provides a method for posture recognition of the human body part to be inspected based on photogrammetry, which captures and calculates the human body part to be inspected in real time through an RGB camera and a depth camera to know the deviation from the optimal position of the X-ray tube theory. In this manner, the medical staff can guide the patient to adjust the posture and position, as much as possible, to coincide with the theoretical optimal position prior to shooting the X-ray. In this manner, the problem of low X-ray image quality caused by the body's standing position or posture deviation is reduced. Of course, while the system of the present invention can quantify the current state of the patient in real time, the human body cannot remain absolutely still, however any slight position and posture deflection will likely not bring a large impact on the quality of the X-ray image. Therefore, it is not required in actual operation to make the patient's current posture 100% coincident with the theoretical optimal position and posture. There is permitted a small error in deviation and if the error is within a preset range, the quality of the obtained X-ray image can be regarded as sufficient to meet the needs of a real diagnosis. Of course, in combination with the guidance of the present invention, should time conditions permit, an operator may spend more time instructing the patient to keep the posture infinitely close to the theoretical optimal position and posture and obtain the highest possible quality X-ray image.

As an example use, if the present invention detects that the current position of the patient deviates from the theoretically optimal position by 5 cm to the left in the horizontal direction, then the medical staff can intuitively instruct the patient to move to the right until the offset error of 5 cm is reduced to a preset range or an acceptable range. The preset range is artificially set and can be adjusted in real time based on different shooting positions. Furthermore, if the current posture of the patient is obviously too hunched, the present invention can obtain the deviation of the patient's current chest posture from the preset posture expressed by Euler angles, so that the medical staff can instruct the patient to adjust the pitch angle, that is, stand upright. Hold your chest up to eliminate the deviation of the current pitch angle, and finally take the X-ray image after the posture and position meet the preset shooting conditions to obtain the ideal X-ray image.

In one example embodiment, the present invention provides a posture recognition method of a human body part to be detected based on photogrammetry, which includes the following steps:
STP100, input the preset position information E₀ of the body part to be detected; the preset position information E₀ including the front and back position, the back and front position, the left lateral position, and the right lateral position. The content of E₀ varies with the body part, and the specific input can be set according to the actual situation.
STP200 adjusts the current part of the human body to be detected within the camera's field of view, and uses the RGB camera and the depth camera to capture the natural image P_{RGB} and the depth image P_{dep} of the part of the human body to be detected respectively.
STP300, based on the YOLO V3 algorithm, according to the natural image P_{RGB} and the depth image P_{dep} obtained in step STP200 to obtain the positioning information E₁ of the current human body to be detected, the center point O of the body part to be detected, and the rectangle surrounded by height h and width w the part to be detected; at the same time, compare the setting information E₁ with the setting information E₀.
STP301: If E₁=E₀, go to step STP400, if E₁≠E₀, go to step STP200;
STP400, to obtain the spatial coordinates of the center point O of the part to be detected;
   Read the pixel coordinates O (x, y) of the center point O of the part to be inspected, and use the RGB camera internal parameter intrinsics_{RGB}, the depth camera internal parameter intrinsics_{depth}, and the transformation matrix extrinsics_{d2c} from the depth camera spatial coordinates to the RGB camera spatial coordinates to output the coordinates O′(x c′, y c′, z c′) of the center point O of the part to be detected in the RGB spatial coordinate system. The above-mentioned camera internal parameters can be directly read and used by the camera without calculation. Among them, the pixel coordinates of the read center point O are known and can be directly obtained from the vision processing library OpenCV, which belongs to the existing technical content and is widely used by those skilled in the art.
STP500 obtains the pixel coordinates of the four corners or vertices P₁, P₂, P₃, and P₄ of the rectangular frame of the part to be detected according to the pixel coordinates O(x, y) of the center point O of the part to be detected.
STP600, by performing regional sampling on the natural image P_{RGB} obtained in step STP200 to fit the reference plane P_{PLA} where the human body surface is located.
STP700, map P₁, P₂, P₃, and P₄ obtained in step STP500 to the reference plane P_{PLA} to obtain a mapping point

$$\begin{matrix}{P}_{1}^{′} ( {x}_{{p}_{1}^{′}} , {y}_{{p}_{1}^{′}} , {z}_{{p}_{1}^{′}} ) {P}_{2}^{′} ( {x}_{{p}_{2}^{′}} , {y}_{{p}_{2}^{′}} , {z}_{{p}_{2}^{′}} ) \\ {P}_{3}^{′} ( {x}_{{p}_{3}^{′}} , {y}_{{p}_{3}^{′}} , {z}_{{p}_{3}^{′}} ) {P}_{4}^{′} ( {x}_{{p}_{4}^{′}} , {y}_{{p}_{4}^{′}} , {z}_{{p}_{4}^{′}} )\end{matrix}$$

STP800, establish a spatial coordinate system with O′ as the origin of the coordinate, as follows:

$$X axis \\ \overline{{O}^{′} x} = \frac{\begin{matrix}( \frac{{x}_{{p}_{2}^{′}} + {x}_{{p}_{1}^{′}} - {x}_{{p}_{1}^{′}} - {x}_{{p}_{1}^{′}}}{2} , \\ \frac{{z}_{{p}_{2}^{′}} + {y}_{{p}_{1}^{′}} - {y}_{{p}_{1}^{′}} - {x}_{{p}_{1}^{′}}}{2} , \frac{{z}_{{p}_{2}^{′}} + {z}_{{p}_{1}^{′}} - {z}_{{p}_{1}^{′}} - {z}_{{p}_{1}^{′}}}{2} )\end{matrix}}{\sqrt{\begin{matrix}{( {x}_{{p}_{2}^{′}} + {x}_{{p}_{1}^{′}} - {x}_{{p}_{1}^{′}} - {x}_{{p}_{1}^{′}} )}^{2} + {( {y}_{{p}_{2}^{′}} + {y}_{{p}_{1}^{′}} - {y}_{{p}_{1}^{′}} - {x}_{{p}_{1}^{′}} )}^{2} + \\ {( {z}_{{p}_{2}^{′}} + {z}_{{p}_{1}^{′}} - {z}_{{p}_{1}^{′}} - {x}_{{p}_{1}^{′}} )}^{2}\end{matrix}}}$$

Take the reference plane PPLAnormal vector \vec{n} as the Z axis, that is \vec{o′}z=\vec{n}; the Y axis can be calculated:

$$\vec{{o}^{′} y} = \vec{{o}^{′} z} \times \vec{{o}^{′} x}$$

Then the unit direction vector \vec{o′x}, \vec{o′y}, \vec{o′z} of the spatial coordinate system O′XYZ of the human body to be detected can be obtained, and the rotation matrix of O′XYZ relative to the coordinate system of x-ray tube can be calculated

$$R = [ \begin{matrix}\vec{{O}^{′} {x}_{x}} & \vec{{O}^{′} {y}_{x}} & \vec{{O}^{′} {z}_{x}} \\ \vec{{O}^{′} {x}_{y}} & \vec{{O}^{′} {y}_{y}} & \vec{{O}^{′} {z}_{y}} \\ \vec{{O}^{′} {x}_{z}} & \vec{{O}^{′} {y}_{z}} & \vec{{O}^{′} {z}_{z}}\end{matrix} ]$$

[symbol]

Then, the Euler angle θx□θy□θz of the spatial coordinate system O′XYZ of the human body to be detected relative to the x-ray tube can be solved□

$${θ}_{x} = {tan}^{- 1} \frac{\vec{{O}^{′} {y}_{z}}}{\vec{{O}^{′} {z}_{z}}} \\ {θ}_{y} = {tan}^{- 1} \frac{- {O}^{′} {x}_{z}}{\sqrt{{\vec{{O}^{′} {y}_{z}}}^{2} + {\vec{{O}^{′} {z}_{z}}}^{2}}} \\ {θ}_{z} = {tan}^{- 1} \frac{\vec{{O}^{′} {x}_{y}}}{\vec{{O}^{′} {x}_{x}}}$$

Finally, the attitude deviation of the human body to be detected relative to the x-ray tube is obtained; among them, if the angle between the vector \vec{n}
   \vec{m}=(0, 0, 1) in the RGB space coordinate system θ>90°, then the direction of the Z axis is −\vec{n}.
      As a preferred calculation method of the present invention, the STP400 obtains the spatial coordinate calculation steps of the center point O of the part to be detected as follows:
STP410, obtain RGB camera internal reference intrinsics_{RGB};
STP420, get the intrinsics_{depth} of the depth camera internal parameters.
STP430, obtain the transformation matrix extrinsics_{d2c} from the depth camera spatial coordinate system to the RGB camera spatial coordinate system.
STP440, get the height H and width W of the depth image P_{dep}; where the start position in the x direction is x_{begin}=0, and the end is x_{end}=W, the start position in the y direction is y_{begin}=0, and the end is y_{end}=H;
STP450, the pixel coordinate D(x_{d}, y_{d}) on the depth map is selected by the dichotomy method, where

$${x}_{d} = \frac{{x}_{begin} + {x}_{end}}{2} \qquad {y}_{d} = \frac{{y}_{begin} + {y}_{end}}{2}$$

STP460, calculate the space coordinate D_{d}′=(x_{d}′, y_{d}′, z_{d}′) corresponding to point D, where

$$[ \begin{matrix}{x}_{d}^{′} \\ {y}_{d}^{′} \\ {z}_{d}^{′} \\ 1\end{matrix} ] = {intrinsics}_{depth}^{- 1} {Z}_{d} [ \begin{matrix}{x}_{d} \\ {y}_{d} \\ 1\end{matrix} ]$$

Calculate the mapping coordinates from the depth camera space coordinate system to the RGB camera space coordinate system D_{c}′=(x_{c}′, y_{c}′, z_{c}′), among them

$$[ \begin{matrix}{x}_{c}^{′} \\ {y}_{c}^{′} \\ {z}_{c}^{′} \\ 1\end{matrix} ] = {extrinsics}_{d 2 c} [ \begin{matrix}{x}_{d}^{′} \\ {y}_{d}^{′} \\ {z}_{d}^{′} \\ 1\end{matrix} ]$$

STP470, convert D_{c}′ to RGB pixel coordinate system D_{c}=(x_{c}, y_{c}), among them

$$[ \begin{matrix}{x}_{c} \\ {y}_{c} \\ 1\end{matrix} ] = \frac{1}{{Z}_{d}} {intrinsics}_{R G B} [ \begin{matrix}{x}_{c}^{′} \\ {y}_{c}^{′} \\ {z}_{c}^{′} \\ 1\end{matrix} ]$$

STP480 will calculate the error Eₐ₀ between the center point O of the part to be detected and Dc in step STP470, where

$${E}_{a 0} = \sqrt{{( {x}_{c} - x )}^{2} + {( {y}_{c} - y )}^{2}}$$

By comparing whether the error Eₐ₀ belongs to the preset error threshold range, if the error Eₐ₀ does not meet the threshold condition, return to step STP450; if the error Eₐ₀ meets the threshold condition, output the space coordinate O of the center point O of the detection part in the RGB camera space coordinate system0 ′(x_{c}′, y_{c}′, z_{c}′).
   Preferably, step STP460 further includes a conversion step between the image unit of any point in the depth image P_{dep} and the standard length sheet, which is specifically as follows:
Calculate the coordinate of point D z_{d}=value(xd, yd)×scale, where value(x_{d}, y_{d}) is the pixel value of point D, and scale is the mapping relationship between the image unit extracted from the depth camera and the standard length unit.
   Preferably, the pixel coordinates of the four vertices P₁, P₂, P₃, and P₄ of the rectangular frame of the detection part in step STP500 are calculated in the following manner:

$$P 1 = ( xw / 2 , y - h / 2 )$$

$$P 2 = ( x + w / 2 , y - h / 2 )$$

$$P 3 = ( x + w / 2 , y + h / 2 )$$

$$P 4 = ( x - w / 2 , y + h / 2 )$$

Among them, h and w are respectively the height and width of the rectangle of the part to be detected.
Preferably, the fitting steps of the reference plane PPLA in step STP600 are as follows:
STP610, with point O as the center, sampling equally spaced up, down, left and right with an interval of

$$d = \frac{1}{N - 1} \frac{min ( w , h )}{2} ,$$

the sampling points are paired in pairs and symmetric left and right, and randomly sample N×N points in the sampling area to obtain the sampling point set S={(x₁₁, y₁₁), (x₁₂, y₁₂), . . . , (X_{NN}, y_{NN})}, transform the sampling point set S into a space coordinate point set T={(x₁₁, y₁₁, z₁₁), (x₁₂, y₁₂, z₁₂), . . . , (x_{NN}, y_{NN}, z_{NN})}; where N≥5;
   As a preferred way, in order to further improve the accuracy, step STP610 also includes the step of correcting the point set T to obtain the corrected point set T′, which specifically includes the following steps:
STP611, take the point T_{m,n}(x_{m,n}, y_{m,n}, z_{m,n}) on the n column in the m row and the point T_{m,N-n}(x_{m,N-n}, y_{m,N-n}, z_{m,N-n}) equidistantly distributed on the N-n column on the other side of the O′ point, and modify them to:
STP612, establishes the reference plane P_{PLA} of step STP620 with the data

$${T}_{m , n}^{′} ( x - \frac{{x}_{m , N - n} - {x}_{m , n}}{2} , \frac{{y}_{m , n} + {y}_{m , N - n}}{2} , z - \frac{{z}_{m , N - n} - {z}_{m , n}}{2} )$$

$${T}_{m , N - n}^{′} ( x + \frac{{x}_{m , N - n} - {x}_{m , n}}{2} , \frac{{y}_{m , n} + {y}_{m , N - n}}{2} , z + \frac{{z}_{m , N - n} - {z}_{m , n}}{2} )$$

of the point set T′ obtained in step STP611.
STP620 uses the point set T or T′ in step STP610 as the data to establish the reference plane P_{PLA} equation Aₓ+B_{y}+C_{z}=D where the human body surface is located, and uses the least squares method to fit the parameters A, B, C, and D;
STP630, The minimum energy equation E_{q} is as follows:

$${E}_{{q}_{( A , B , C , D )}} = {\sum }_{i = 1}^{N \times N} {[ D - ( A {x}_{i} + B {y}_{i} + C {z}_{i} ) ]}^{2}$$

Among them, i={1, 2, . . . , N×N}, xᵢ, yᵢ, zᵢ are the space coordinates of the i-th point respectively;
STP640, through iterative calculation of gradient descent method, makes E_{q} obtain the minimum parameter M=[A, B, C, D], and parameter M is the optimal parameter required by the P_{PLA} equation of the reference plane.
Preferably, the mapping method for mapping P₁, P₂, P₃, and P₄ onto the reference plane P_{PLA} in step STP700 includes the following steps:
STP710, the width w_{d} and height ha of the known flat panel imaging detector;
STP720, set the boundary vertices of the reference plane P_{PLA} of the human body surface to U₁, U₂, U₃, U₄, then there are

$${U}_{1} = ( x - {w}_{d} , y - {h}_{d} , \frac{D - A ( x - {w}_{d} ) - B ( y - {h}_{d} )}{C} ) ,$$

$${U}_{2} = ( x + {w}_{d} , y - {h}_{d} , \frac{D - A ( x + {w}_{d} ) - B ( y - {h}_{d} )}{C} ) ,$$

$${U}_{3} = ( x + {w}_{d} , y + {h}_{d} , \frac{D - A ( x + {w}_{d} ) - B ( y + {h}_{d} )}{C} ) ,$$

$${U}_{4} = ( x - {w}_{d} , y + {h}_{d} , \frac{D - A ( x - {w}_{d} ) - B ( y + {h}_{d} )}{C} ) ,$$

STP730, set the starting position of the reference plane P_{PLA} X direction x_{begin}=x−w_{d}, the end position x_{end}=x+w_{d}, set the y direction. The starting position of y_{begin}=y−h_{d}, end position y_{end}=y+h_{d};
STP740, using dichotomy to select the plane space coordinates U(x_{f}, y_{f}, z_{f}) of the human body surface, where

$${x}_{f} = ( {x}_{begin} + {x}_{end} ) / 2 ,$$

$${y}_{f} = ( {y}_{begin} + {y}_{end} ) / 2 ,$$

$${z}_{f} = ( D - {Ax}_{f} - {By}_{f} ) / C ;$$

STP750, convert U to RGB pixel coordinate system U′=(x′_{f}, y′_{f})

$$[ \begin{matrix}{x}_{f}^{′} \\ {y}_{f}^{′} \\ 1\end{matrix} ] = \frac{1}{{z}_{f}} {intrinsics}_{R G B} [ \begin{matrix}{x}_{f} \\ {y}_{f} \\ {z}_{f} \\ 1\end{matrix} ]$$

Among them, intrinsics_{RGB} is the internal reference of the RGB camera;
STP760, calculate the error Eₐ₁ between Pₙ,n∈{1, 2, 3, 4} and U′ as:

$${E}_{a 1} = \sqrt{{( {x}_{f} - {x}_{n} )}^{2} + {( {y}_{f} - {y}_{n} )}^{2}}$$

Output Pₙ by judging the magnitude of the errors Eₐ₁ and Eₘᵢₙ, where n∈{1, 2, 3, 4}, xₙ, yₙ are the pixel values of Pₙ in the RGB pixel coordinate system; map Pₙ to the reference plane P_{PLA} The mapping point Pₙ′(x_{Pn}′, y_{Pn}′, z_{Pn}′) on the mapping point, the judgment method is as follows:
STP761, if Eₐ₁≤Eₘᵢₙ, then U′ is the optimal approximation point of Pₙ point, and U is the mapping of Pₙ point on the reference plane P_{PLA}, and step STP760 is performed;
STP762, if Eₐ₁>Eₘᵢₙ, then determine the size of xf and xn, the size of y_{f} and yₙ, if xₙ<x_{f}, x_{begin}=x_{f}, otherwise x_{end}=x_{f}, similarly if yₙ<y_{f}, y_{begin}=y_{f}, otherwise y_{end}=y_{f}, return to step STP740.

By acquiring the position and posture of the target part of the human body in real time, the present invention can provide feedback of the difference between the patient's current posture and the theoretical best shooting posture to the medical staff in real time, so that the medical staff can intuitively know how the patient should adjust the current position and posture. In this manner, an operator may quickly align the patient's target position within the theoretical optimal position range, thereby obtaining high-quality X-ray images at one time, eliminating the problem of low X-ray image quality caused by posture and distance problems.

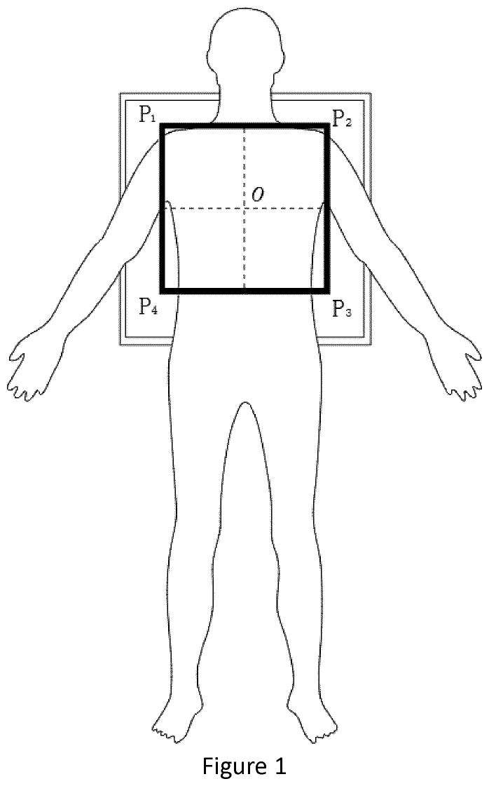

In order to provide an example of the present invention, including example technical effects and application convenience, one example preferred implementation of the present invention will be described below. The example is explained in detail in conjunction with the specific shooting location and is illustrated with the assistance of FIG. 1, which shows a schematic of an X-ray of a person's chest.

This example embodiment provides a method for recognizing the position and posture of the human body part to be detected based on photogrammetry, which is used to recognize the position and posture of the human body part to be detected in real time during X-ray shooting. The deviation between the best position and posture is preset to facilitate real-time adjustment, the patient's position is quickly adjusted, and a single high-quality X-ray is taken once, eliminating the need to rely on the operator's experience to guide the patient in the correct position.

This example embodiment is described by taking a chest radiograph as an example, which specifically includes the following steps:
STP100, input chest preset position information E₀; the preset position information E₀ described in this embodiment includes front and back positions, back and front positions, left lateral positions and right lateral positions. According to different body parts, the content of the positioning information E₀ may be less than or more than the positioning described in this embodiment. Specifically, it can be set and input according to the actual situation.
STP200, adjust the human chest to the camera's field of view, and use the RGB camera and the depth camera to capture the natural image P_{RGB} and the depth image P_{dep} of the chest, respectively.
STP300, based on the YOLO V3 algorithm, according to the natural image P_{RGB} and depth image P_{dep} obtained in step STP200 to obtain the current chest position information E₁, the chest center point O, and the rectangular chest surrounded by height h and width w; at the same time, the setting information E₁ is compared with the setting information E₀ in step STP100.

If E₁=E₀, go to step STP400, if E₁≠E₀, go to step STP200.
STP400, get the spatial coordinates of the center point O of the chest. Read the pixel coordinates O(x, y) of the center point O of the chest, and use the RGB camera internal parameter intrinsics_{RGB}, the depth camera internal parameter intrinsics_{depth}, and the transformation matrix extrinsics_{d2c} from the depth camera space coordinates to the RGB camera space coordinates to output the coordinates O′(x c′, y c′, z c′) of the center point O of the chest in the RGB space coordinate system. Among them, the pixel coordinates of the center point O are known and can be directly obtained through the vision processing library OpenCV.

The steps for calculating the spatial coordinates of the chest center point O are as follows:
STP410, get the intrinsics_{RGB} of the RGB camera.
STP420, get the intrinsics_{depth} of the depth camera internal parameters.
STP430, obtain the transformation matrix extrinsics_{d2c} from the depth camera space coordinate system to the R G B camera space coordinate system.
STP440, get the height H and width W of the depth image P_{dep}. The start position in the x direction is x_{begin}=0, the end position is x_{end}=W, the start position in the y direction is y_{begin}=0, and the end position is y_{end}=H.
STP450, using dichotomy to select pixel coordinates D(x_{d}, y_{d}) on the depth map, where

$${x}_{d} = \frac{{x}_{begin} + {x}_{e n d}}{2} {y}_{d} = \frac{{y}_{begin} + {y}_{e n d}}{2}$$

STP460, calculate the space coordinate Dd corresponding to point D′=(x_{d}′, y_{d}′, z_{d}′), where

$$[ \begin{matrix}{x}_{d}^{′} \\ {y}_{d}^{′} \\ {z}_{d}^{′} \\ 1\end{matrix} ] = {intrinsics}_{depth}^{- 1} {Z}_{d} [ \begin{matrix}{x}_{d} \\ {y}_{d} \\ 1\end{matrix} ]$$

Calculate the mapping coordinates from the depth camera space coordinate system to the RGB camera space coordinate system D_{c}′=(x_{c}′, y_{c}′, z_{c}′),

$$[ \begin{matrix}{x}_{c}^{′} \\ {y}_{c}^{′} \\ {z}_{c}^{′} \\ 1\end{matrix} ] = {ex trinsics}_{d 2 c} [ \begin{matrix}{x}_{d}^{′} \\ {y}_{d}^{′} \\ {z}_{d}^{′} \\ 1\end{matrix} ]$$

This step also includes the conversion step between the image unit of any point in the depth image P_{dep} and the standard-length unit, which is specifically as follows:
Calculate the coordinate of point D zₓ=value(x_{d}, y_{d})×scale, where value(x_{d}, y_{d}) is the pixel coordinate of point D, and scale is the mapping relationship between the image unit extracted from the depth camera and the standard length unit.
STP470, convert D_{c} to RGB pixel coordinate system D_{c}=(x_{c}, y_{c}):

$$[ \begin{matrix}{x}_{c} \\ {y}_{c} \\ 1\end{matrix} ] = \frac{1}{{Z}_{d}} {intrinsics}_{R G B} [ \begin{matrix}{x}_{c}^{′} \\ {y}_{c}^{′} \\ {z}_{c}^{′} \\ 1\end{matrix} ]$$

STP480, calculate the error Eₐ₀ between the chest center point O of the part to be detected and Dc in step STP470, where

$${E}_{a 0} = \sqrt{{( {x}_{c} - x )}^{2} + {( {y}_{c} - y )}^{2}}$$

By comparing whether the error Eₐ₀ belongs to the preset error threshold range, if the error Eₐ₀ does not meet the threshold condition, return to step STP450; if the error Eₐ₀ meets the threshold condition, output the space coordinate O of the center point O of the detection part in the RGB camera space coordinate system0 ′(x_{c}′, y_{c}′, z_{c}′).

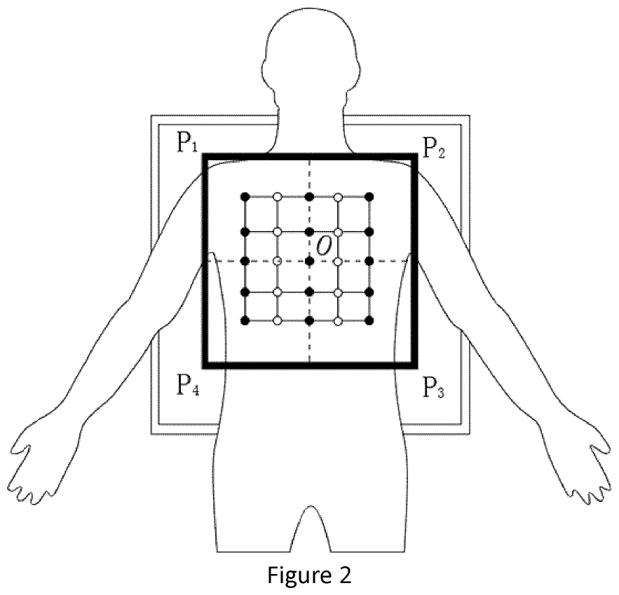

FIG. 2 is representative of Steps 500 to 800 below.
STP500 obtains the pixel coordinates of the four vertices P₁, P₂, P₃, and P₄ of the chest rectangle according to the pixel coordinates O(x, y) of the center point O of the chest, as shown in FIG. 1. The pixel coordinates of the vertices P₁, P₂, P₃, and P₄ are calculated in the following way:

$$P 1 = ( x - w / 2 , y - h / 2 )$$

$$P 2 = ( x + w / 2 , y - h / 2 )$$

$$P 3 = ( x + w / 2 , y + h / 2 )$$

$$P 4 = ( x - w / 2 , y + h / 2 )$$

Among them, h and w are respectively the height and width of the rectangle of the chest to be detected.
STP600, by performing region sampling on the natural image P_{RGB} obtained in step STP200 to fit the reference plane P_{PLA} where the chest surface is located; the fitting steps of the reference plane P_{PLA} are as follows:
STP610, with point O as the center, sampling equally spaced up, down, left and right with an interval of

$$d = \frac{1}{N - 1} \frac{min ( w , h )}{2} ,$$

the sampling points are paired in pairs and symmetric left and right, and randomly sample N×N points in the sampling area to obtain the sampling point set S={(x₁₁, y₁₁), (x₁₂, y₁₂), . . . , (X_{NN}, y_{NN})}, transform the sampling point set S into a space coordinate point set T={(x₁₁, y₁₁, z₁₁), (x₁₂, y₁₂, z₁₂), . . . , (x_{NN}, y_{NN}, z_{NN})}; where N≥5;

In order to further improve the accuracy, step STP610 also includes the step of correcting the point set T to obtain the corrected point set T′, which specifically includes the following steps:
STP611, take the points T_{m,n}(x_{m,n}, y_{m,n}, z_{m,n}) on the n column in the m row and the points T_{m,N-n}(x_{m,N-n}, y_{m,N-n}, z_{m,N-n}) equidistantly distributed on the N-n column on the other side of the O point, and modify them to:

$${T}_{m , n}^{′} ( x - \frac{{x}_{m , N - n} - {x}_{m , n}}{2} , \frac{{y}_{m , n} + {y}_{m , N - n}}{2} , z - \frac{{z}_{m , N - n} - {z}_{m , n}}{2} )$$

$${T}_{m , N - n}^{′} ( x + \frac{{x}_{m , N - n} - {x}_{m , n}}{2} , \frac{{y}_{m , n} + {y}_{m , N - n}}{2} z , + \frac{{z}_{m , N - n} - {z}_{m , n}}{2} )$$

STP612, establish the reference plane P_{PLA} of step STP620 with the data of the point set T′ obtained in step STP611.
STP620, using the corrected point set T′ in step STP610 as the data to establish the reference plane P_{PLA} equation Aₓ+B_{y}+C_{z}=D where the chest surface is located, and fitting the parameters A, B, C, D by the least square method.
STP630, establish the minimum energy equation E_{q} as follows:

$${E}_{{q}_{( A , B , C , D )}} = {\sum }_{i = 1}^{N \times N} {[ D - ( A {x}_{i} + B {y}_{i} + C {z}_{i} ) ]}^{2}$$

wherein, i={1, 2, . . . , N×N}, xᵢ, yᵢ, zᵢ are the space coordinates of the i-th point respectively.
STP640, through iterative calculation of gradient descent method, makes E_{q} obtain the minimum parameter M=[A, B, C, D], and parameter M is the optimal parameter required by the P_{PLA} equation of the reference plane.
STP700, mapping P₁, P₂, P₃, and P₄ obtained in step STP500 to the reference plane P_{PLA} to obtain mapping points

$${P}_{1}^{′} ( {x}_{{p}_{1}^{′}} , {y}_{{p}_{1}^{′}} , {z}_{{p}_{1}^{′}} ) {P}_{2}^{′} ( {x}_{{p}_{2}^{′}} , {y}_{{p}_{2}^{′}} , {z}_{{p}_{2}^{′}} )$$

$${P}_{3}^{′} ( {x}_{{p}_{3}^{′}} , {y}_{{p}_{3}^{′}} , {z}_{{p}_{3}^{′}} ) {P}_{4}^{′} ( {x}_{{p}_{4}^{′}} , {y}_{{p}_{4}^{′}} , {z}_{{p}_{4}^{′}} )$$

The mapping method for mapping P1, P2, P3 and P4 onto the reference plane P_{PLA} includes the following steps:
STP710, the width w_{d} and height h_{d} of the known detector.
STP720, set the boundary vertices of the reference plane P_{PLA} of the chest surface to U₁, U₂, U₃, U₄, then there are:

$${U}_{1} = ( x - {w}_{d} , y - {h}_{d} , \frac{D - A ( x - {w}_{d} ) - B ( y - {h}_{d} )}{C} ) ,$$

$${U}_{2} = ( x + {w}_{d} , y - {h}_{d} , \frac{D - A ( x + {w}_{d} ) - B ( y - {h}_{d} )}{C} ) ,$$

$${U}_{3} = ( x + {w}_{d} , y + {h}_{d} , \frac{D - A ( x + {w}_{d} ) - B ( y + {h}_{d} )}{C} ) ,$$

$${U}_{4} = ( x - {w}_{d} , y + {h}_{d} , \frac{D - A ( x - {w}_{d} ) - B ( y + {h}_{d} )}{C} ) ,$$

STP730, set the starting position of the reference plane P_{PLA} X direction x_{begin}=x−w_{d}, the end position x_{end}=x+w_{d}, set the y direction. The starting position of y_{begin}=y−h_{d}, end position y_{end}=y+h_{d};
STP740, using dichotomy to select the plane space coordinates U(x_{f}, y_{f}, z_{f}) of the chest surface, where

$${x}_{f} = ( {x}_{begin} + {x}_{end} ) / 2 ,$$

$${y}_{f} = ( {y}_{begin} + {y}_{end} ) / 2 ,$$

$${z}_{f} = ( D - {Ax}_{f} - {By}_{f} ) / C ;$$

STP750, convert U to RGB pixel coordinate system U′=(x′_{f}, y′_{f})

$$[ \begin{matrix}{x}_{f}^{′} \\ {y}_{f}^{′} \\ 1\end{matrix} ] = \frac{1}{{z}_{f}} {intrinsics}_{R G B} [ \begin{matrix}{x}_{f} \\ {y}_{f} \\ {z}_{f} \\ 1\end{matrix} ]$$

wherein, intrinsics_{RGB} is the internal reference of the RGB camera.
STP760, calculate the error Eₐ₁ between Pₙ,n∈{1, 2, 3, 4} and U′ as:

$${E}_{a 1} = \sqrt{{( {x}_{f} - {x}_{n} )}^{2} + {( {y}_{f} - {y}_{n} )}^{2}}$$

Output Pₙ by judging the magnitude of the errors Eₐ₁ and Eₘᵢₙ, where n∈{1, 2, 3, 4}, xₙ, yₙ are the pixel values of Pₙ in the RGB pixel coordinate system; map Pₙ to the reference plane P_{PLA} The mapping point Pₙ′(x_{Pn}′, y_{Pn}′, z_{Pn}′) on the mapping point, the judgment method is as follows:
STP761, if Eₐ₁≤Eₘᵢₙ, then U′ is the optimal approximation point of Pₙ point, and U is the mapping of Pₙ point on the reference plane P_{PLA}, and step STP760 is performed.
STP762, if Eₐ₁>Eₘᵢₙ, then determine the size of xf and xn, the size of y_{f} and yₙ, if xₙ<x_{f}, x_{begin}=x_{f}, otherwise x_{end}=x_{f}, similarly if yₙ<y_{f}, y_{begin}=y_{f}, otherwise yend=y_{f}, return to step STP740.
STP800, a spatial coordinate system is established with O′ as the origin of the coordinate, and the details are as follows:

### X Axis

$$\vec{{O}^{′} x} = \frac{\begin{matrix}( \frac{{x}_{{p}_{\text{?}}^{′}} + {x}_{{p}_{\text{?}}^{′}} - {x}_{{p}_{\text{?}}^{′}} - {x}_{{p}_{\text{?}}^{′}}}{2} , \\ \frac{{z}_{{p}_{\text{?}}^{′}} + {y}_{{p}_{\text{?}}^{′}} - {y}_{{p}_{\text{?}}^{′}} - {x}_{{p}_{\text{?}}^{′}}}{2} , \frac{{z}_{{p}_{\text{?}}^{′}} + {z}_{{p}_{\text{?}}^{′}} - {z}_{{p}_{\text{?}}^{′}} - {z}_{{p}_{\text{?}}^{′}}}{2} )\end{matrix}}{\sqrt{\begin{matrix}{( {x}_{{p}_{\text{?}}^{′}} + {x}_{{p}_{\text{?}}^{′}} - {x}_{{p}_{\text{?}}^{′}} - {x}_{{p}_{\text{?}}^{′}} )}^{2} + \\ {( {y}_{{p}_{\text{?}}^{′}} + {y}_{{p}_{\text{?}}^{′}} - {y}_{{p}_{\text{?}}^{′}} - {x}_{{p}_{\text{?}}^{′}} )}^{2} + {( {z}_{{p}_{\text{?}}^{′}} + {z}_{{p}_{\text{?}}^{′}} - {z}_{{p}_{\text{?}}^{′}} - {x}_{{p}_{\text{?}}^{′}} )}^{2}\end{matrix}}}$$

$$\text{?} \text{indicates text missing or illegible when filed}$$

Take the reference plane P_{PLA} normal vector \vec{n} as the Z axis, that is \vec{o′}z=\vec{n}; the Y axis can be calculated:

$$\vec{{o}^{′} y} = \vec{{o}^{′} z} \times \vec{{o}^{′} x}$$

Then the unit direction vector \vec{o′x}, \vec{o′y}, \vec{o′z} of the spatial coordinate system O′XYZ of the human body to be detected can be obtained, and the rotation matrix of O′XYZ relative to the coordinate system of the x-ray tube can be calculated

$$R = [ \begin{matrix}\vec{{O}^{′} {x}_{x}} & \vec{{O}^{′} {y}_{x}} & \vec{{O}^{′} {z}_{x}} \\ \vec{{O}^{′} {x}_{y}} & \vec{{O}^{′} {y}_{y}} & \vec{{O}^{′} {z}_{y}} \\ \vec{{O}^{′} {x}_{z}} & \vec{{O}^{′} {y}_{z}} & \vec{{O}^{′} {z}_{z}}\end{matrix} ]$$

[symbol]

Then, the Euler angle θx□θy□θz of the spatial coordinate system O′XYZ of the chest to be detected relative to the x-ray tube can be solved□

$${θ}_{x} = {tan}^{- 1} \frac{\vec{{O}^{′} {y}_{z}}}{\vec{{O}^{′} {z}_{z}}} \\ {θ}_{y} = {tan}^{- 1} \frac{- \vec{{O}^{′} {x}_{z}}}{\sqrt{{{\vec{{O}^{′} {y}_{z}}}^{}}^{2} + {{\vec{{O}^{′} {z}_{z}}}^{}}^{2}}} \\ {θ}_{z} = {tan}^{- 1} \frac{\vec{{O}^{′} {x}_{y}}}{\vec{{O}^{′} {x}_{x}}}$$

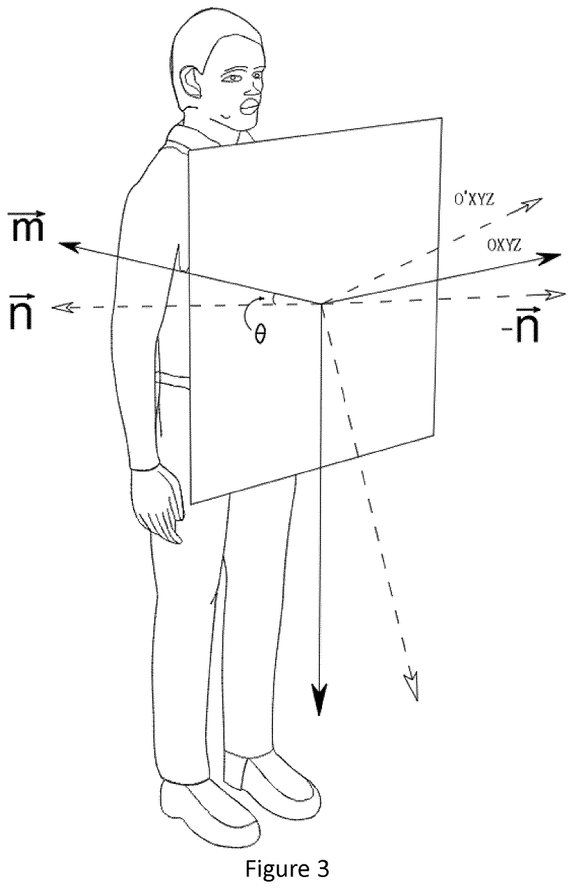

Finally, the attitude deviation of the chest to be detected relative x-ray tube is obtained; among them, if the angle between the vector \vec{n} and the OXY normal vector \vec{m}=(0, 0, 1) in the RGB space coordinate system θ>90°, then the direction of the Z axis is −\vec{n}, as shown in FIG. 3.

It should be emphasized that the RGB space coordinate system in this application and the space coordinate system where x-ray tube on the X-ray equipment is located are the same coordinate system or two coordinate systems without spatial deflection with translation vectors. If the space translation vector exists, then the system software can eliminate the translation vector through the algorithm. That is to say, no matter where the depth camera, RGB camera and the x-ray tube are installed and how many translation vectors exist, then the calculation process in the x-ray tube may be used as the coordinate center of the space vector to express the space coordinates of any other space coordinate system, such as the depth camera, RGB camera, and any space coordinate of the space coordinate system where the center of the human body is located. The purpose of obtaining the deflection Euler angle between two space coordinate systems is to establish the relative position relationship between the two objects in the space coordinate system. What is embodied in the present invention is that when performing X-ray shooting, medical operators can grasp the position of the patient's target shooting part in real time, thereby guiding the patient to adjust the corresponding posture, so that the target body part can be included in the preset shooting within the range to obtain the ideal X-ray image.

After the posture deviation is obtained, the deviation between the real-time position of the patient's chest and the theoretical preset position is displayed on the existing display, so that the medical operator can clearly guide the patient how to adjust the posture, so that the adjustment is fast, the position is accurate, and the standard position is used for reference, which eliminates the uncertainty caused by the subjective cognition of medical operators.

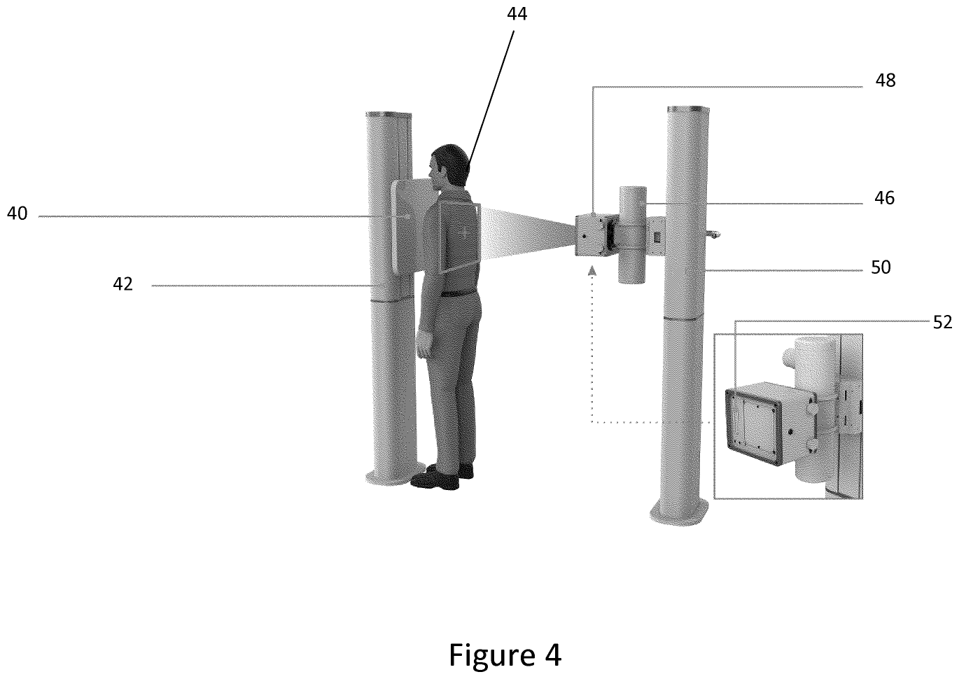

FIG. 4 illustrates a sample schematic of use of the invention in a scan of a chest of a human patient. A flat panel imaging detector 40 is attached to a detector stand 42. A patient 44 stands in front of the imaging detector 40 awaiting a scan of the chest of the patient. The x-ray tube 46 is attached to the tube stand 50 and connected to the collimator 48 which is directed toward the patient's chest. The collimator 48 contains the RGB camera and depth image camera 52. Although the arrangement has been demonstrated with a scan of a chest of a patient, the system can be adapted and modified for an x-ray of any body part.

It will be appreciated by one skilled in the art that variants can exist in the above-described arrangements and applications.

Following from the above description, it should be apparent to those of ordinary skill in the art that, while the methods and apparatuses herein described constitute exemplary embodiments of the present invention, the invention described herein is not limited to any precise embodiment and that changes may be made to such embodiments without departing from the scope of the invention as defined by the claims. Consequently, the scope of the claims should not be limited by the preferred embodiments set forth in the examples but should be given the broadest interpretation consistent with the description as a whole.

Likewise, it is to be understood that it is not necessary to meet any or all of the identified advantages or objects of the invention disclosed herein in order to fall within the scope of any claims, since the invention is defined by the claims and since inherent and/or unforeseen advantages of the present invention may exist even though they may not have been explicitly discussed herein.

What is claimed is:

1. A method for posture recognition of a human body part to be imaged with an X-ray machine, comprising the steps of:
inputting preset position information of the body part to be detected;
adjusting the body part into a field of view of a camera;
using the camera to capture a natural image of the body part;
using a depth camera to capture a depth image of the body part;
establishing a spatial attitude deviation of the body part relative to a spatial coordinate system where the X-ray tube is located;
determining a desired position for the body part based on the spatial attitude deviation;
providing the desired position for the body part in real time to an operator; and
adjusting the body part to a new position based on the desired position;
wherein the camera is an RGB camera.

2. The method of claim 1 further comprising the step of obtaining positioning information of the body part, determining a center point of the body part; and determining a rectangle surrounding the center point.

3. The method of claim 2 further comprising the step of obtaining the spatial coordinates of the center point, and using the internal parameters of the camera and internal parameters of the depth camera to create a transformation matrix to transform the center point to an RGB space coordinate system.

4. The method of claim 3 further comprising the step of obtaining spatial coordinates of corners of the rectangle.

5. The method of claim 4 further comprising the step of performing regional sampling on the natural image to fit a reference plane where the human body surface is located.

6. The method of claim 5 further comprising the step of mapping the corners of the rectangle to the reference plane to obtain a mapping point.

7. The method of claim 6 further comprising the step of establishing a new spatial coordinate system with O′ as the origin of the coordinate using the equation:
$$\vec{{O}^{′} x} = \frac{\begin{matrix}( \frac{{x}_{{p}_{\text{?}}^{′}} + {x}_{{p}_{\text{?}}^{′}} - {x}_{{p}_{\text{?}}^{′}} - {x}_{{p}_{\text{?}}^{′}}}{2} , \frac{{z}_{{p}_{\text{?}}^{′}} + {y}_{{p}_{\text{?}}^{′}} - {y}_{{p}_{\text{?}}^{′}} - {x}_{{p}_{\text{?}}^{′}}}{2} , \\ \frac{{z}_{{p}_{\text{?}}^{′}} + {z}_{{p}_{\text{?}}^{′}} - {z}_{{p}_{\text{?}}^{′}} - {z}_{{p}_{\text{?}}^{′}}}{2} )\end{matrix}}{\sqrt{\begin{matrix}{( {x}_{{p}_{\text{?}}^{′}} + {x}_{{p}_{\text{?}}^{′}} - {x}_{{p}_{\text{?}}^{′}} - {x}_{{p}_{\text{?}}^{′}} )}^{2} + \\ {( {y}_{{p}_{\text{?}}^{′}} + {y}_{{p}_{\text{?}}^{′}} - {y}_{{p}_{\text{?}}^{′}} - {x}_{{p}_{\text{?}}^{′}} )}^{2} + {( {z}_{{p}_{\text{?}}^{′}} + {z}_{{p}_{\text{?}}^{′}} - {z}_{{p}_{\text{?}}^{′}} - {x}_{{p}_{\text{?}}^{′}} )}^{2}\end{matrix}}}$$
$$\text{?} \text{indicates text missing or illegible when filed}$$
wherein the mapping point is notated as
$${P}_{1}^{′} ( {x}_{{p}_{1}^{′}} , {y}_{{p}_{1}^{′}} , {z}_{{p}_{1}^{′}} ) {P}_{2}^{′} ( {x}_{{p}_{2}^{′}} , {y}_{{p}_{2}^{′}} , {z}_{{p}_{2}^{′}} )$$
$${P}_{3}^{′} ( {x}_{{p}_{3}^{′}} , {y}_{{p}_{3}^{′}} , {z}_{{p}_{3}^{′}} ) {P}_{4}^{′} ( {x}_{{p}_{4}^{′}} , {y}_{{p}_{4}^{′}} , {z}_{{p}_{4}^{′}} )$$

8. The method of claim 7 further comprising the step of determining a spatial coordinate system of the human body part to be detected, determining a rotation matrix relative to a coordinate system of an x-ray tube, determining the Euler angle of the spatial coordinate system of the human body to be detected, and determining an attitude deviation of the human body part to be detected relative to the x-ray tube.

9. The method of any one of claims 1 to 8 wherein the preset position information includes front and back positions, back and front positions, left lateral position and right lateral position information.

10. The method of any one of claims 1 to 9 further comprising the step of detecting an error in the new position relative to the desired position.

11. The method of claim 10 further comprising the step of ignoring the error if the error belongs to a preset error threshold range.

12. The method of claim 4 wherein the corners of the rectangle are calculated as follows:
$$P 1 = ( x - w / 2 , y - h / 2 )$$
$$P 2 = ( x + w / 2 , y - h / 2 )$$
$$P 3 = ( x + w / 2 , y + h / 2 )$$
$$P 4 = ( x - w / 2 , y + h / 2 )$$
wherein h and w are respectively height and width of the rectangle.

13. The method of claim 6 wherein the step of mapping the corners comprises the following steps:
setting boundary vertices of the reference plane of the human body surface to U₁, U₂, U₃, U₄, as follows
$${U}_{1} = ( x - {w}_{d} , y - {h}_{d} , \frac{D - A ( x - {w}_{d} ) - B ( y - {h}_{d} )}{C} ) , {U}_{2} = ( x + {w}_{d} , y - {h}_{d} , \frac{D - A ( x + {w}_{d} ) - B ( y - {h}_{d} )}{C} ) , {U}_{3} = ( x + {w}_{d} , y + {h}_{d} , \frac{D - A ( x + {w}_{d} ) - B ( y + {h}_{d} )}{C} ) , {U}_{4} = ( x - {w}_{d} , y + {h}_{d} , \frac{D - A ( x - {w}_{d} ) - B ( y + {h}_{d} )}{C} ) :$$
wherein w_{d} is the width and h_{d} is the height of a flat panel imaging detector.

14. The method of claim 7, further comprising the steps of:
taking a reference plane P_{PLA} normal vector \vec{n} as a Z axis, that is \vec{o′}z=\vec{n};
calculating the Y axis as:
$$\vec{{o}^{′} y} = \vec{{o}^{′} z} \times \vec{{o}^{′} x}$$
detecting a unit direction vector \vec{o′x}, \vec{o′y}, \vec{o′z} of the spatial coordinate system O′XYZ of the body part;
calculating a rotation matrix of O′XYZ relative to the spatial coordinate system of the x-ray tube as follows:
$$R = [ \begin{matrix}\vec{{O}^{′} {x}_{x}} & \vec{{O}^{′} {y}_{x}} & \vec{{O}^{′} {z}_{x}} \\ \vec{{O}^{′} {x}_{y}} & \vec{{O}^{′} {y}_{y}} & \vec{{O}^{′} {z}_{y}} \\ \vec{{O}^{′} {x}_{z}} & \vec{{O}^{′} {y}_{z}} & \vec{{O}^{′} {z}_{z}}\end{matrix} ]$$ [symbol]
detecting a Euler angle θx□θy□θz of a spatial coordinate system O′XYZ of the body part relative to the x-ray tube as follows□
$${θ}_{x} = {tan}^{- 1} \frac{\vec{{O}^{′} {y}_{z}}}{\vec{{O}^{′} {z}_{z}}} \\ {θ}_{y} = {tan}^{- 1} \frac{- \vec{{O}^{′} {x}_{z}}}{\sqrt{{{\vec{{O}^{′} {y}_{z}}}^{}}^{2} + {{\vec{{O}^{′} {z}_{z}}}^{}}^{2}}} \\ {θ}_{z} = {tan}^{- 1} \frac{\vec{{O}^{′} {x}_{y}}}{\vec{{O}^{′} {x}_{x}}}$$
detecting an attitude deviation of the body part relative to the x-ray tube;
wherein, if an angle between vector \vec{n} and OXY normal vector \vec{m}=(0, 0, 1) in the RGB space coordinate system is greater than 90°, then the direction of the Z axis is −\vec{n}.

15. The method of claim 3, wherein the step of obtaining the spatial coordinates of the center point O of the body part comprises the steps of:
obtaining an internal reference intrinsics_{RGB} of the camera;
obtaining an intrinsic depth intrinsics_{depth} of the depth camera;
obtaining a transformation matrix extrinsics_{d2c} from a space coordinate system of the depth camera to a coordinate system of the camera.
obtaining height H and width W of the depth image, where a start position in the x direction is x_{begin}=0, and an end position in the x direction is x_{end}=W, and a start position in the y direction is y_{begin}=0, and an end position in the y direction is y_{end}=H;
selecting a pixel coordinate D(x_{d}, y_{d}) on a depth map by a dichotomy method, where
$${x}_{d} = \frac{{x}_{begin} + {x}_{end}}{2} {y}_{d} = \frac{{y}_{begin} + {y}_{end}}{2}$$
calculating a space coordinate D_{d}′=(x_{d}′, y_{d}′, z_{d}′) corresponding to point D, and conversion steps between an image unit of any point in the depth image and a standard length unit as follows:
calculating coordinates of D point z_{d}=value(x_{d}, y_{d})×scale, where value(x_{d}, y_{d}) is a pixel value of D point, and scale is a mapping relationship between the image unit from the depth camera and the standard length unit;
$$[ \begin{matrix}{x}_{d}^{′} \\ {y}_{d}^{′} \\ {z}_{d}^{′} \\ 1\end{matrix} ] = {intrinsics}_{d e p t h}^{- 1} {Z}_{d} [ \begin{matrix}{x}_{d} \\ {y}_{d} \\ 1\end{matrix} ]$$
calculating mapping coordinates from the depth camera space coordinate system to the camera space coordinate system D_{c}′=(x_{c}′, y_{c}′, z_{c}′), wherein
$$[ \begin{matrix}{x}_{c}^{′} \\ {y}_{c}^{′} \\ {z}_{c}^{′} \\ 1\end{matrix} ] = {extrinsics}_{d 2 c} [ \begin{matrix}{x}_{d}^{′} \\ {y}_{d}^{′} \\ {z}_{d}^{′} \\ 1\end{matrix} ]$$
convert D_{c}′ to RGB pixel coordinate system D_{c}=(x_{c}, y_{c}), wherein
$$[ \begin{matrix}{x}_{c} \\ {y}_{c} \\ 1\end{matrix} ] = \frac{1}{{Z}_{d}} {intrinsics}_{RGB} [ \begin{matrix}{x}_{c}^{′} \\ {y}_{c}^{′} \\ {z}_{c}^{′} \\ 1\end{matrix} ]$$
calculating an error Ea0 between the center point O of the body part and Dc, wherein
$${E}_{a 0} = \sqrt{{( {x}_{c} - x )}^{2} + {( {y}_{c} - y )}^{2}}$$
comparing whether the error Eₐ₀ belongs to a preset error threshold condition, if the error Eₐ₀ does not meet the preset error threshold condition, return to the step of selecting the pixel coordinate, if the error Eₐ₀ meets the preset error threshold condition, output the space coordinate O of the center point O of the body part in the camera space coordinate system0 ′(x_{c}′, y_{c}′, z_{c}′).

16. The method of claim 5 further comprising the steps of
sampling equally spaced up, down, left and right with an interval of
$$d = \frac{1}{N - 1} \frac{min ( w , h )}{2} ,$$
and with point O as the center,
pairing the sampling points in pairs and symmetrically left and right;
randomly sampling N×N points in a sampling area to obtain a sampling point set S={(x₁₁, y₁₁), (x₁₂, y₁₂), . . . , (X_{NN}, y_{NN})};
transforming the sampling point set S into a space coordinate point set T={(x₁₁, y₁₁, z₁₁), (x₁₂, y₁₂, z₁₂), . . . , (x_{NN}, y_{NN}, z_{NN})}; where N≥5;
using the point set T to establish a reference plane P_{PLA} equation Aₓ+B_{y}+C_{z}=D where the human body surface is located,
using least squares method to fit the parameters A, B, C, and D;
establishing a minimum energy equation E_{q} as follows:
$${E}_{{q}_{( A , B , C , D )}} = {\sum }_{i = 1}^{N \times N} {[ D - ( A {x}_{i} + B {y}_{i} + C {z}_{i} ) ]}^{2}$$
wherein, i={1, 2, . . . , N×N}, xᵢ, yᵢ, zᵢ are the space coordinates of the i-th point respectively;
using iterative calculation of gradient descent method to make E_{q} obtain a minimum parameter M=[A, B, C, D], wherein parameter M is an optimal parameter required by the P_{PLA} equation of the reference plane.

17. The method of claim 16, further comprising the steps of:
setting a starting position of reference plane P_{PLA} in an X direction with x_{begin}=x−w_{d}, and an end position of x_{end}=x+w_{d};
setting a y direction with a starting position of y_{begin}=y−h_{d} and end position of y_{end}=y+h_{d};
using dichotomy to select plane space coordinates U(x_{f}, y_{f}, z_{f}) of the human body surface, where
$${x}_{f} = ( {x}_{begin} + {x}_{end} ) / 2 ,$$
$${y}_{f} = ( {y}_{begin} + {y}_{end} ) / 2 ,$$
$${z}_{f} = ( D - {Ax}_{f} - {By}_{f} ) / C ;$$
converting U to RGB pixel coordinate system U′=(x′_{f}, y′_{f}) as follows:
$$[ \begin{matrix}{x}_{f}^{′} \\ {y}_{f}^{′} \\ 1\end{matrix} ] = \frac{1}{{z}_{f}} {intrinsics}_{RGB} [ \begin{matrix}{x}_{f} \\ {y}_{f} \\ {z}_{f} \\ 1\end{matrix} ]$$
wherein, intrinsics_{RGB} is an internal reference of the RGB camera;
calculating an error Eₐ₁ between Pₙ,n∈{1, 2, 3, 4} and U′ as:
$${E}_{a 1} = \sqrt{{( {x}_{f} - {x}_{n} )}^{2} + {( {y}_{f} - {y}_{n} )}^{2}}$$
outputting Pₙ by judging magnitude of errors Eₐ₁ and Eₘᵢₙ, wherein n∈{1, 2, 3, 4}, and xₙ, yₙ are the pixel values of Pₙ in the RGB pixel coordinate system;
mapping Pₙ to the reference plane P_{PLA} with mapping point Pₙ′(x_{Pn}′, y_{Pn}′, z_{Pn}′)
using judgment method as follows:
if Eₐ₁≤Eₘᵢₙ, then U′ is optimal approximation point of the Pₙ point, and U is the mapping of Pₙ point on the reference plane P_{PLA};
if Eₐ₁>Eₘᵢₙ, then determine size of x_{f} and xₙ and the size of y_{f} and yₙ,
if xₙ<x_{f}, x_{begin}=x_{f}, otherwise x_{end}=x_{f}, similarly if yₙ<y_{f}, y_{begin}=y_{f}, otherwise y_{end}=y_{f}, then return to the step of using dichotomy to select the plane space coordinates.

18. The method of claim 16 further comprising the steps of
correcting the point set T to obtain a corrected point set T′ as follows:
$${T}_{m , n}^{′} ( x - \frac{{x}_{m , N - n} - {x}_{m , n}}{2} , \frac{{y}_{m , n} + {y}_{m , N - n}}{2} , z - \frac{{z}_{m , N - n} - {z}_{m , n}}{2} ) \\ {T}_{m , N - n}^{′} ( x + \frac{{x}_{m , N - n} - {x}_{m , n}}{2} , \frac{{y}_{m , n} + {y}_{m , N - n}}{2} , z + \frac{{z}_{m , N - n} - {z}_{m , n}}{2} )$$
establishing the reference plane P_{PLA} with the point set T′.

19. An X-ray system for imaging a body part, comprising:
an RGB camera aligned towards the body part and configured to capture a natural image of the body part;
a depth camera aligned towards the body part and configured to capture a depth image of the body part;
an X-ray tube aligned towards the body part;
a controller connected to the RGB camera and depth camera;
wherein controller establishes a spatial attitude deviation of the body part relative to a spatial coordinate system where the X-ray tube is located, determines a desired position for the body part based on the spatial attitude deviation and provides the desired position for the body part in real time to an operator.
